(19) United States
(12) Patent Application Publication
(10) Pub. No.: US 2005/0022259 A1
(43) Pub. Date: Jan. 27, 2005

(54) Transgenic animal model for cancer and stem cells

(76) Inventors: Steven C. Pruitt, Williamsville, NY (US); Alexander Y. Maslov, Williamsville, NY (US); Tara A. Barone, Snyder, NY (US); Robert J. Plunkett, Orchard Park, NY (US)

Correspondence Address: HODGSON RUSS LLP, ONE M & T PLAZA, SUITE 2000, BUFFALO, NY, 14203-2391, US

(21) Appl. No.: 10/842,683

(22) Filed: May 10, 2004

Related U.S. Application Data
(60) Provisional application No. 60/468,904, filed on May 8, 2003.

(51) Int. Cl. A01K 67/027; C12N 5/06; C12N 5/08

(52) U.S. Cl. 800/18; 435/366; 435/354

(57) ABSTRACT

The present invention provides methods and compositions for identifying stem cells and/or cancer cells in non-human animals. The compositions include vectors comprising elements suitable for integration into a licensing factor gene such that a cell harboring the chromosome into which the vector elements have integrated expresses a reporter transgene. The methods comprise the detection of stem and/or cancer cells by detecting the expression of the reporter transgene.

This application claims the priority of U.S. Provisional Application Ser. No. 60/468,904, filed on May 8, 2003, the disclosure of which is incorporated herein by reference.

This invention was supported by grant numbers AG20946 and AG19863 from the National Institutes of Health. The Government has certain rights in the invention.

### FIELD OF THE INVENTION

The present invention relates generally to the detection of stem and/or cancer cells in whole animal models.

### BACKGROUND OF THE INVENTION

“Licensing factors” are proteins which are required for the initiation of DNA replication through the formation of pre-replicative complexes at replication origins. They also have a role in preventing multiple rounds of DNA synthesis during a single S-phase. Such complexes are established in G1 and their presence maintains the origin in a state in chromatin that is competent for initiation of replication, in essence “licensing” the origin for use in the subsequent S-phase. While present in G1 nuclei, licensing factors are consumed during the replication process. When the nuclear envelope breaks down during mitosis, the nuclear store of licensing factor is replenished from cytoplasmic stores, allowing DNA replication in the next cell cycle. Licensing factors are expressed in proliferation competent cells regardless of whether they are actively cycling, but transcription of licensing factor genes is turned off upon withdrawal of the cells from the cell cycle during differentiation. Based on the fact that licensing factors are expressed early in G1 of the cell cycle due to their role in marking replication origins for use in the subsequent S-phase, licensing factors should be present in a high percentage of cells that are resting in G1, such as cycling proliferative progenitors and slowly cycling stem cell populations. Immunohistological detection of various licensing factors such as the mini-chromosome maintenance (Mcm) family, together with origin recognition complex (ORC) and Cdc6, detects a significantly higher fraction of cycling cells in tumors and stem cells than are detected using conventional proliferation markers such as PCNA and Ki67. However, immunohistological methods do not allow for detection of stem or cancer cells in living animals, such as in whole body imaging.

Thus, there is a need for an animal model and method of detecting licensing factor expression characteristic of stem and/or cancer cells. However, there is no convenient system by which the expression of licensing factors can be detected in living cells. The present invention meets this need.

### SUMMARY OF THE INVENTION

The present invention provides methods and compositions for detecting stem cells and/or cancer cells and their progeny in non-human animals. The compositions include vectors comprising elements suitable for integration into a licensing factor gene such that a cell harboring the chromosome into which the vector elements have integrated expresses a reporter transgene.

Accordingly, in one embodiment, the invention provides a transgene reporter vector suitable for integrating a reporter transgene into a licensing factor gene wherein expression of the licensing factor gene results in expression of the reporter transgene.

In another embodiment, the invention provides a recombinase vector suitable for integrating a recombinase into a licensing factor gene wherein expression of the licensing factor gene results in expression of the recombinase, which in turn induces expression of the reporter transgene.

The invention further comprises in various embodiment methods for using the vectors to generate transgenic cells and non-human animals, and further provides for the detection of stem and/or cancer cells or their progeny in the transgenic animals by detecting the expression of a reporter transgene.

### BRIEF DESCRIPTION OF THE FIGURES

FIG. 1A is a graphical representation of the reporter transgene vector mcm2IresEGFP.

FIG. 1B is a graphical representation of the mouse Mcm2 site where the reporter transgene vector depicted in FIG. 1A will homologously recombine.

FIG. 1C is a graphical representation of the mouse Mcm2 gene after homologous recombination with the reporter transgene vector depicted in FIG. 5A.

FIG. 2A is a low magnification bright field micrograph of corneal slice image of EGFP in the sub-ventricular zone of 4 week old Mcm2-EGFP transgenic mice.

FIG. 2B is the same field shown in FIG. 2A using fluorescence microscopy.

FIG. 2C is a higher magnification fluorescence micrograph showing the boxed regions indicated in FIGS. 2A and 2B.

FIG. 3A Fluorescence micrograph of spleen section in negative control mouse (no EGFP transgene reporter).

FIG. 3B Bright field microgrpah of spleen section in negative control mouse (no EGFP transgene reporter).

FIG. 3C Fluorescence micrograph of spleen section in mouse expressing the EGFP transgene reporter.

FIG. 3D Bright field micrograph of spleen section in mouse expressing the EGFP transgene reporter.

FIG. 4A is an image from living brain tissue showing the SVZ region of the lateral ventricle from an Mcm2 EGFP transgenic animal.

FIG. 4B is a higher of magnification of FIG. 4A.

FIGS. 4C-4E show co-localization in transgenic mice of EGFP (FIG. 4C, green, cytoplasmic) with Mcm2 (FIG. 4D, red, nuclear).

FIG. 4E is an overlay of the images in FIG. 4C and FIG. 4D with DAPI nuclear stain (blue) of the same cells.

FIGS. 4F-4H show the same stains as for 4C-4E for the hippocampus.

FIG. 4F shows living skeletal muscle from an Mcm2-EGFP transgenic animal.

FIG. 5A is a graphical representation of the recombinase vector Cre-ERT2.

FIG. 5B is a graphical representation of the mouse Mcm2 site where the Cre-ERT2 vector depicted in FIG. 5A will homologously recombine.

FIG. 5C is a graphical representation of the mouse Mcm2 gene after homologous recombination with the Cre-ERT2 vector depicted in FIG. 5A.

FIG. 6A is a graphical representation of (−) TAM (tamoxefin) cultures showing only red fluorescent cells.

FIG. 6B is a graphical representation of (+) TAM cultures having cells co-expressing both hcRed and EGFP.

### DETAILED DESCRIPTION OF THE INVENTION

The present invention provides methods and compositions for identifying stem cells and/or cancer cells in non-human animals. The compositions include vectors comprising elements suitable for integration into a licensing factor gene such that a cell harboring the chromosome into which the vector elements have integrated expresses a reporter transgene.

By “licensing factor” it is meant proteins that form part of pre-replicative complexes prior to the initiation of chromosomal replication and result in chromatin being “licensed” for replication in S phase of the cell cycle. Exemplary licensing factors contemplated by the present invention include but are not limited to origin-recognition complex (ORC), Cdc6, Cdt1, and the minichromosome maintenance (MCM) family of proteins Mcm2, Mcm3, Mcm4, Mcm5, Mcm6 and Mcm7.

“Transgene” means any piece of DNA which is inserted by artifice into a cell and becomes part of the genome of the organism either by integration or as an extrachromosomal element. “Transgenes” as used herein further means polynucleotides comprising protein coding regions alone or in addition to expression control elements such as internal ribosome entry sequences.

Accordingly, in one embodiment, the present invention provides a transgene reporter in a vector suitable for integrating the reporter transgene into a licensing factor gene in the genome of a non-human animal wherein expression of the licensing factor gene results in expression of the reporter transgene.

In another embodiment, the invention provides a recombinase vector suitable for integrating a recombinase transgene into a licensing factor gene in the genome of a non-human animal wherein expression of the licensing factor gene results in expression of the recombinase transgene, which in turn induces expression of the reporter transgene, which is also present in the genome in a gene other than the licensing factor gene.

Any reporter transgene can be used in the present invention. In one embodiment, a fluorescent protein is used as the reporter. Any fluorescent protein can be used. For example, green fluorescent proteins (“GFPs”) of cnidarians are suitable fluorescent proteins for use in the fluorescent indicators.

GFPs have been isolated from the Pacific Northwest jellyfish, Aequorea victoria, the sea pansy, Renilla reniformis, and Phialidium gregarium. A variety of Aequorea-related GFPs having useful excitation and emission spectra have been engineered by modifying the amino acid sequence of a naturally occurring GFP from Aequorea victoria. Other genes encoding fluorescent proteins can be also be used as reporter transgenes, such as, for example, yellow fluorescent protein from Vibrio fischeri strain Y-1, Peridinin-chlorophyll binding protein from the dinoflagellate Symbiodinium, phycobiliproteins from marine cyanobacteria such as Synechococcus, e.g., phycoerythrin and phycocyanin, oat phytochromes from oat reconstructed with phycoerytrobilin, β-galactosidae, alkaline phosphatase, or β-lactamase.

Nucleic acids encoding fluorescent proteins can be obtained by methods known in the art. For example, a nucleic acid encoding a fluorescent protein can be isolated by polymerase chain reaction of cDNA from A. victoria using primers based on the DNA sequence of A. victoria green fluorescent protein. PCR methods are well known and described in, for example, U.S. Pat. No. 4,683,195 and Sambrook et al, “Molecular Cloning: A Laboratory Manual” (2001) Cold Spring Harbor Laboratory Press; 3rd edition.

The vectors of the present invention may also comprise various expression control sequences including promoters, enhancers, transcription terminators, start or stop codons, splicing signals, elements for maintenance of the correct reading frame of a gene to permit proper translation of the mRNA, and elements such as internal ribosome entry sites (‘Ires”), leader sequences and other untranslated sequences. Moreover, control sequences can be considered as part of a gene.

By “promoter” is meant a nucleotide sequence sufficient to direct transcription. Also included in the invention are those promoter elements which are sufficient to render promoter-dependent gene expression controllable for cell-type specific, tissue-specific, or inducible by external signals or agents. Such elements may be located in the 5′ or 3′ regions of the gene and may be constitutive and/or inducible. For example, when cloning in mammalian cell systems, promoters derived from the genome of mammalian cells or from mammalian viruses may be used in the vectors of the present invention. Promoters produced by recombinant DNA: or synthetic techniques may also be used to provide for transcription of the nucleic acid sequences of the invention. Depending on the vector utilized, any of a number of suitable transcription and translation elements, including constitutive and inducible promoters, promoters endogenous to the organism in which the vectors of the present invention can be used, transcription enhancer elements, transcription terminators, etc. may be used in the expression vector according to methods well known to one of skill in the art.

In a particular embodiment, a transgene reporter vector of the present invention comprises from 5′ to 3′ a first region of a licensing factor gene, a stop codon, an internal ribosome entry site, a reporter transgene, and a second region of the licensing factor gene. The vector may also comprise various restriction enzyme cleavage sites anywhere in the vector. The first and second regions of the licensing factor gene are homologous to a licensing factor gene endogenous to a non-human animal. Thus, the transgene reporter vector has the capacity to homologously recombine with a chromosome having the same or similar licensing factor gene as that included in the vector.

In another embodiment, a method is provided for using the reporter transgene vector. The method comprises the steps of homologously recombining the reporter transgene vector with a licensing gene in a cell and detecting the expression of the reporter transgene. As will be apparent to those skilled in the art, after the vector has homologously recombined with the target gene, the vector sequences between the 3′ end of the first region and 5′ end of the second region of the licensing factor gene are inserted into the endogenous licensing factor gene. This results in a chromosomal configuration whereby transcription from an endogenous licensing factor gene promoter will also drive transcription of the inserted vector sequence. Further, when the inserted vector sequence includes a stop codon 3′ to the first licensing factor region, followed by an internal ribosome entry site and a transgene reporter coding sequence, translation of the transgene reporter is facilitated by the internal ribosome entry site. In this way, homologous recombination with the reporter transgene vector of the present invention will result in cells that express the reporter transgene concomitantly with expression of the licensing factor gene. Further, by using the method of the present invention, detection of the reporter transgene results in the simultaneous detection of cells expressing the endogenous licensing factor. In one embodiment, the reporter transgene is enhanced green fluorescent protein (EGFP) and the licensing factor is the mouse Mcm2 gene.

In another embodiment of the present invention, a recombinase vector is provided that enables detection of cells expressing licensing factor genes and further enables detection of their progeny cells, irrespective of whether the licensing factor gene is being expressed in the progeny cells. Accordingly, a recombinase vector is provided which comprises from 5′ to 3′ a first region of a licensing factor gene, a stop codon, an internal ribosome entry site, a recombinase transgene, and a second region of the licensing factor gene. With respect to the first and second regions, any region of a licensing factor gene that directs insertion of the transgene into the licensing factor gene such that the trasgene is expressed under control of the licensing factor gene promoter is sufficient.

The vector may also comprise various restriction enzyme cleavage sites anywhere in the vector. The first and second regions of the licensing factor gene are homologous to a licensing factor gene endogenous to a non-human animal. The recombinase vector thus has the capacity to homologously recombine with a chromosome having the target licensing factor gene. When the vector has homologously recombined with the target gene, the result is that the vector sequences between the 3′ end of the first region and 5′ end of the second region of the licensing factor gene are inserted into the endogenous licensing factor gene. As will be apparent to those skilled in the art, this results in a chromosomal configuration whereby transcription from the endogenous licensing factor gene promoter will also drive transcription of the inserted coding region for a recombinase transgene.

Further, when the inserted vector sequence includes a stop codon 3′ to the licensing factor coding region, followed by an internal ribosome entry site and a recombinase transgene coding sequence, translation of the recombinase transgene is facilitated by the internal ribosome entry site. In this way, homologous recombination with the vector of the present invention will result in cells that express the recombinase transgene concomitantly with expression of the licensing factor.

Any recombinase can be used in the present invention. Examples of suitable recombinases include, Cre recombinase, tamoxifen inducible Cre recombinase, and Flp. A preferred recombinase is an inducible recombinase, such as tamoxifen inducible Cre recombinase. Examples of non-CRE recombinases include, but are not limited to FLP recombinase of the 2μ plasmid of Saccharomyces cerevisiae, the recombination sites recognized by the resolvase family, and the recombination site recognized by transposase of Bacillus thruingiensis.

In the Cre-lox system, the recombination sites are referred to as “lox sites” and the recombinase is referred to as “Cre”. Cre is a site-specific DNA recombinase isolated from bacteriophage P1. The lox (locus of crossover in P1) is 34 bp in length. It has two 13 bp inverted repeats flanking an 8 bp non-palindromic core sequence. The core sequence determines the direction of the lox site. Cre-mediated recombination between two directly repeated lox sites results in an irreversible excision of the intervening sequence. When lox sites are in parallel orientation (i.e., in the same direction), then Cre catalyzes a deletion of the intervening polynucleotide sequence. When lox sites are in the opposite orientation, the Cre recombinase catalyzes an inversion of the intervening polynucleotide sequence. Tamoxifen inducible Cre recombinase is a fusion of Cre recombinase to the ligand binding domain of the human estrogen receptor (ER), resulting in a tamoxifen-dependent Cre recombinase that is activated by tamoxifen.

In another embodiment, a method is provided for using the recombinase vector of the present invention. The method comprises the steps of homologously recombining the recombinase vector with a licensing gene in a cell wherein the cell also harbors a recombinase dependant reporter transgene and detecting the expression of the reporter transgene.

“Recombinase dependent” means that in order for the reporter transgene to be expressed, a recombinase expressed from a recombinase transgene must first catalyze a recombination that allows the transgene to be expressed. However, once the recombinase catalyzes the genetic alteration, the reporter transgene can continue to be expressed whether or not the recombinase is present. For example, continuous expression of the reporter transgene can be achieved by transcription of the reporter transgene from a constitutive promoter. In a preferred embodiment, the recombinase vector comprises a Cre recombinase transgene coding region and the reporter transgene is EGFP.

In another embodiment, a transgenic system such as a transgenic cell or transgenic animal is provided in which is present a Cre recombinase transgene coding region under the control of a licensing factor gene promoter and a Cre dependent reporter transgene that is permanently expressed only in the case of Cre catalyzed recombination of DNA elements, such as LOX sites. Recombinations leading to transgene reporter expression occur in the cell expressing the licensing factor gene and mark the cell's progeny by expression of the reporter transgene regardless of whether the progeny continue to express the licensing factor gene that caused the initial expression of Cre. This methodology is an effective means of following the fate of a cell in the forward direction. In a preferred embodiment, transient activation of Cre recombinase by an activator such as tamoxefin enables Cre to catalyze a genetic rearrangement that results in EGFP expression from a constitutive Pgk promoter as well as its progeny. The reporter transgene that is activated upon Cre expression can be obtained commercially, such as from The Jackson Laboratory (Bar Harbor, ME). In one embodiment, the reporter transgene is carried on a vector such as a Pgk-lox-STOP-lox-EGFP vector. In another embodiment, transgenic cells or transgenic organisms already harboring an integrated Pgk-lox-STOP-lox-EGFP sequence can be used in combination with a vector of the present invention or crossed with a non-human animal having Cre under the control of a licensing gene factor.

As will be apparent to those skilled in the art, the construction and use of the vectors of the present invention require transfection or transformation of cells at various times. Transformation of a host cell with recombinant DNA may be carried out by conventional techniques as are well known to those skilled in the art. Where the host is prokaryotic, such as E. coli, competent cells which are capable of DNA uptake can be prepared from treated by known methods using for example, CaCl₂ or MgCl₂. Transformation can also be performed after forming a protoplast of the host cell or by electroporation.

When the host is a eukaryote, methods of transfection of DNA such as calcium phosphate co-precipitates, conventional mechanical procedures such as microinjection, electroporation, insertion of a plasmid encased in liposomes, or virus vectors may be used. Another method is to use a eukaryotic viral vector, such as simian virus 40 (SV40) adenovirus, vaccinia virus, or bovine papilloma virus, to transiently infect or transform eukaryotic cells and express a protein. Methods of stable transfer, meaning that the foreign DNA is maintained in the host for a suitable time, are known in the art. Further, methods by which DNA homologously recombine with the host chromosome are also known.

Eukaryotic systems, and preferably mammalian expression systems, allow for proper post-translational modifications of expressed mammalian proteins to occur. Eukaryotic cells which possess the cellular machinery for proper processing of the primary transcript, glycosylation, phosphorylation, and if necessary, secretion of the gene product are preferably used as host cells for the expression of the transgene reporter.

The present invention further provides methods for using the vectors of the invention to engineer embryonic stem (ES) cells, cell lines and transgenic non-human animals from the ES cells.

Transgenes can be used to create transgenic organisms in a variety of ways known to those skilled in the art. For example, embryonal target cells at various developmental stages can be used to introduce transgenes. Different methods are used depending on the stage of development of the embryonal target cell. The zygote is the best target for micro-injection. In the mouse, the male pronucleus reaches the size of approximately 20 micrometers in diameter which allows reproducible injection of 1-2 pl of DNA solution. The use of zygotes as a target for gene transfer is advantageous in that in most cases the injected DNA will be incorporated into the host gene before the first cleavage (Brinster et al., Proc. Natl. Acad. Sci USA 82:4438-4442, 1985). As a consequence, all cells of the transgenic non-human animal will carry the incorporated transgene. This will in-general also be reflected in the efficient transmission of the transgene to offspring of the founder since 50% of the germ cells will harbor the transgene. Viral infection can also be used to introduce transgene into a non-human animal e.g., retroviral, adenoviral or any other RNA or DNA viral vectors using known techniques.

Another method for introduction of transgenes into a non-human animal is referred to as gene trapping (Skames et al., 1992, Genes Dev., 6:903-18; Durick et al., 1999, Genome Res., 9:1019-1025; Pruitt et al. 1992, Development 116:573-583). The basic gene trap vector contains a splice acceptor site immediately upstream of a reporter gene and a selectable marker. When a gene trap vector integrates into an intron of a gene, the reporter is situated such that it becomes under the transcriptional control of the trapped gene's promoter, the trapped gene being defined by integration of the reporter into the trapped gene's intron. When the trapped gene is transcribed, a fusion transcript is generated between upstream exons and the reporter gene.

Accordingly, in one embodiment a vector of the present invention may comprises sequences representing gene-trapping functions and target gene modification for insertion of a reporter transgene or a recombinase transgene into a licensing factor gene. Such a vector may additionally comprise a series of termination codons in all three reading frame to ensure that the endogenous transcript codon does not occlude the internal ribosome entry site, an internal ribosome entry site, a nucleotide sequence encoding a reporter (such as one capable of directly or indirectly producing fluorescence), a poly-adenylation signal to terminate transcription, a promoter sequence, and a selectable marker. The gene trap vector may also comprise a DNA sequence encoding a recombinase transgene. Because gene trapping involves the random integration of transgenes into the genome, a percentage of such events will lead to trapping of licensing factor genes.

Yet another method for introduction of transgenes into a non-human animal makes use of the potential of embryonic stem cells (ES cells) to create chimeric animals. Embryonic stem cells are derived from the inner cell mass (ICM) of blastocysts and are totipotent cells which are capable of developing into all cell lineages, including germ cells, when introduced into an embryo. As used herein, “embryo” includes developmental stages wherein ES are injected into diploid blastocysts or aggregated with morulae.

ES cells can be isolated from blastocysts and then established as permanent, cell lines wherein they can be genetically manipulated. In view of this ability, they constitute an effective tool for modifying the mammalian and particularly the mouse genome by being introduced into the animals, for example, by means of controlled mutations or other genetic modifications. For examples of methods for producing transgenic animals, see U.S. Pat. No. 6,492,575 to Wagner, et al. for “Method for developing transgenic mice”, the disclosure of which is incorporated herein by reference. The isolation of ES cells from blastocysts, the establishing of ES cell lines and their subsequent cultivation are carried out by conventional methods as described, for example, by Doetchmann et al., (1985) J. Embryol. Exp. Morph. 87, 27-45; Li et al., (1992) Cell 69, 915-926. The cultivation of ES cells can be carried out using methods such as those described in Donovan et al (1997, Transgenic Animals, Generation and Use, Ed. L. Houdebine, pp 179-187, Harwood Academic Publishers). Generally, transgenes can be efficiently introduced into the ES cells using standard methods known from the literature for in vitro transfer of DNA into mammalian cells, such as electroporation or methods based on receptor-mediated endocytosis.

Once transfected, the DNA integrates by homologous recombination into the ES genome. Accordingly, in one embodiment the present invention provides ES cells that have homologously combined with a vector of the present invention. Such ES cells can thereafter be combined with blastocysts from a nonhuman animal. This produces a chimeric embryo, which constitutes a mixture of the two embryos. These embryos are then transferred into a pseudo-pregnant mouse which acts as a foster mother. The chimeric offspring obtained have cells, including germ line cells, which originate from one of the two original embryos. In this way, a genetic modification introduced in ES cells by homologous recombination can be introduced in the germ line of a chimeric mouse and be transmitted to generate progeny that are either heterozygous or homozygous for the transgene.

In a particular embodiment the method of the present invention comprises generating a chimeric non-human animal embryo from an ES cell having a recombinase transgene integrated into a licensing factor gene. In this method, the, ES cells are used in the formation of an early developmental stage of the non-human animal suitable-for implantation into a host animal for development, such as a blastocyst or an embryo. The blastocyst or embryo comprising the ES cells is implanted into a host non-human animal for development to obtain a first chimeric non-human animal. The first chimeric non-human animal is crossed with a second chimeric non-human animal having an integrated reporter transgene that requires the activity of the recombinase to induce expression of the reporter transgene. Progeny animals from the cross will have cells that comprise the recombinase transgene integrated into a licensing factor gene, and the reporter transgene. When the licensing factor gene is expressed, the cell is considered “licensed to divide”, and the recombinase transgene is also expressed. When the recombinase is “activated”, meaning it is translocated to the nucleus, the recombinase can reconfigure DNA in the cell such that the reporter transgene can be expressed.

In one embodiment, the recombinase transgene encodes Cre recombinase or a modified Cre recombinase that can be activated by the effect of an “activator” such as tamoxefin. Accordingly, cells expressing the reporter transgene can be identified by detecting the expression of the reporter transgene. As will be described more fully by way of the Examples below, expression of the reporter transgene as detected by the method of the present invention correlates highly with detection of tissues wherein stem cells and/or cancer cells are present.

Expression of reporter transgenes can be detected by a variety of methods well known to those skilled in the art. For example, fluorescence in a sample can be measured using a fluorimeter or any machine capable of measuring fluorescence. In one embodiment, detection is by fluorescently activated cell sorting (FACS) which allows recovery of cells expressing the reporter transgene. In general fluorescence is detected by application of excitation radiation from an excitation source having a first wavelength which passes through excitation optics. The excitation optics cause the excitation radiation to excite the sample. In response, fluorescent proteins in the sample emit radiation which has a wavelength that is different from the excitation wavelength. Collection optics then collects the emission from the sample. Other means of measuring fluorescence can also be used with the invention. For example, fluorescence in living tissue can be detected using fluorescence microscopy or whole body fluorescence imaging.

In a further embodiment, the compositions and methods of the invention can be used in screening assays to determine whether a test agent (e.g., a drug, a chemical or a biologic) alters a property of cells in a transgenic non-human animal of the present invention. In one embodiment, the ability of a test compound or agent to modulate the activity of a licensing factor gene is tested in chimeric or transgenic animals. For example, in in vivo assays such as whole animal imaging, cells possessing transgene reporter constructs of the present invention are exposed to the test compound and the effect on the expression of the reporter transgene, such as a change in fluorescence, in the animal can be determined. Typically, the difference in the expression of the reporter transgene with and without the test agent having been administered is calibrated against standard measurements and/or a control animal to which the test agent has not been administered to yield an absolute amount of reporter activity. This provides a method for screening for compounds which can modulate cellular events such as the expression of a licensing factor gene.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

### EXAMPLE 1

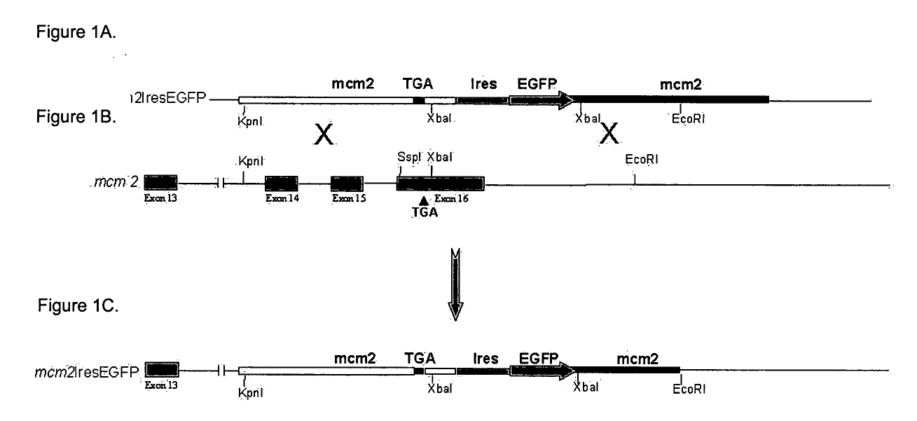

This example demonstrates the construction of the reporter transgene vector mcm2IresEGFP useful in the method of the present invention. The vector as shown in FIG. 1A “targets” the licensing factor gene Mcm2 shown in FIG. 1B. The vector comprises an Ires-EGFP sequence 3′ to the termination codon (TGA) and 5′ to the polyadenylation signal of the endogenous Mcm2 gene. In order to facilitate homologous recombination with the endogenous Mcm2 gene (“targeting”), approximately 8 kbp of 5′ Mcm2 gene sequence and approximately 1 kbp of 3′ Mcm2 gene sequence are included surrounding the insertion site. A constitutive promoter is not required in this particular embodiment because site specific integration of the construct results in expression of the EGFP gene from the endogenous licensing factor promoter. In this particular embodiment, it can be seen from FIG. 1C that homologous recombination of the vector with the Mcm2 gene results in a bi-cistronic transcript such that a single mRNA encodes both the Mcm2 and the EGFP coding regions. Translation of this message results in functional Mcm2. Translation of the EGFP coding regions is achieved by initiation of translation on the Ires “internal ribsosome entry site” 3′ to the start of the EGFP coding regions. Accordingly, the Mcm2 promoter drives expression of both the Mcm2 gene and the EGFP. As will be apparent to those skilled in the art, the same strategy can be used to drive the expression of any gene according to methods known to those skilled in the art.

### EXAMPLE 2

This example demonstrates the efficacy of the reporter transgene vector described in Example 1 for generating transgenic ES cells and the use of the ES cells for the generation of transgenic mice. W4 ES cells having a SV129 mouse background were electroporated with the reporter transgene vector and selected for neomycin resistance. Colonies were picked, expanded and DNA from individual clones was tested by Southern blotting for correct targeting into the Mcm2 gene and confirmed that the reporter transgene vector described in Example 1A correctly targeted Mcm2 gene as shown in FIG. 1A-C.

Chimeric mice were then generated from the ES cells by blastocyst injection into C57 black 6 embryos by implantation into a pseudo-pregnant mouse using standard techniques as described above and further described in Hogen et al., (1994) Manipulating the Mouse Embryo: A laboratory manual. Cold Spring Harbor Laboratory Press, pp. 253-290. 11 chimeric mice were obtained and their chimerism indicated by agouti coat color. These first generation animals were raised to maturity and transmitted the transgene to their offspring to produce transgenic mice as confirmed by Southern blotting.

### EXAMPLE 3

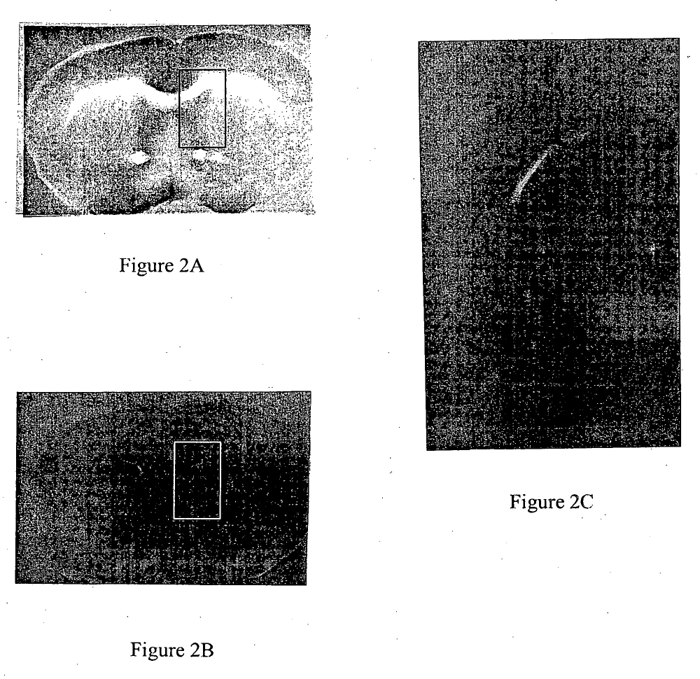

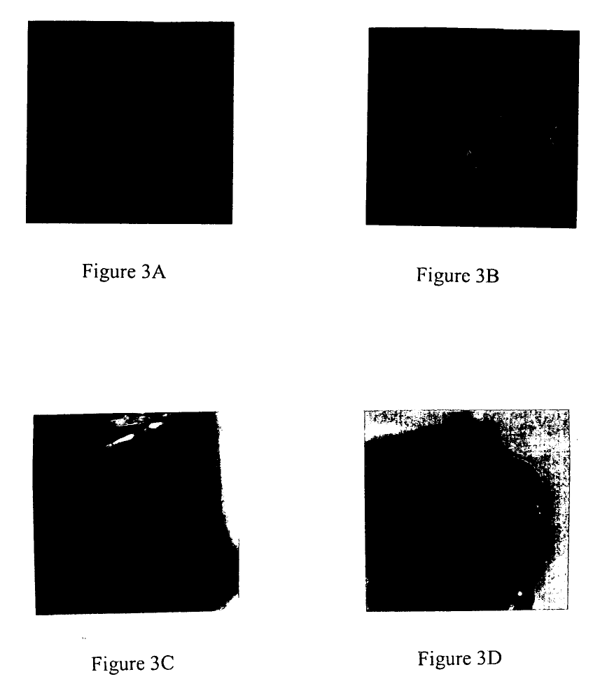

This Example demonstrates that the transgenic mice described in Example 2 provide a means of assaying stem cells and/or cancer growth by detecting the integrated reporter gene. For example, the whole mount preparations shown in FIGS. 2A-C and 3A-3D demonstrate detectable expression of the integrated EGFP. In FIG. 2A, is a low magnification bright field micrograph of corneal slices of the brain of a 2 month old transgenic mouse. FIG. 2B shows expression of EGFP in the sub-ventricular zone of the lateral ventricle using fluorescence stereomicroscopy in the same sample as in FIG. 2A. FIG. 2C is a higher magnification fluorescence micrograph showing the boxed regions indicated in FIGS. 2A and 2B. FIG. 3A shows a fluorescence micrograph of spleen section in negative control mouse having no EGFP transgene reporter. Similarly, FIG. 3B shows a bright field micrograph of spleen section in negative control mouse. In contrast, FIG. 3C. shows a fluorescence micrograph of spleen section in mouse expressing the EGFP transgene reporter in the germinal zone, while FIG. 3D shows a bright field micrograph of spleen section in mouse expressing the EGFP transgene reporter.

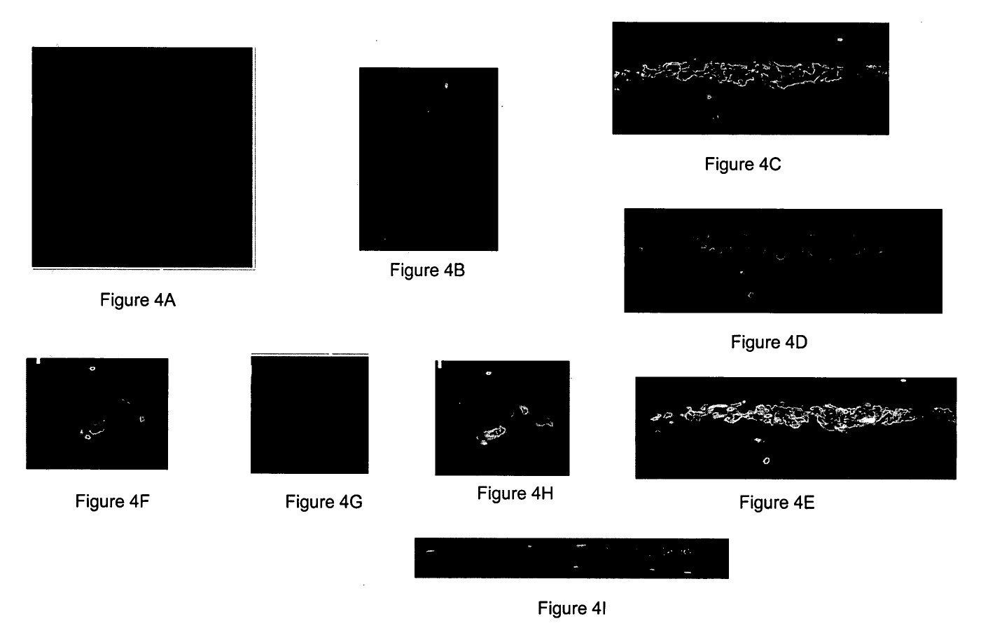

FIGS. 4A-I further demonstrate that the Mcm2-EGFP transgenic mice as described in Example 2 appropriately regulate EGFP expression from the Mcm2 gene. FIGS. 4A and 4B are lower and higher magnification images from living brain tissue showing the SVZ region of the lateral ventricle from an Mcm2 EGFP transgenic animal. FIGS. 4C-4E show co-localization in transgenic mice of EGFP (FIG. 4C, green, cytoplasmic) with Mcm2 (FIG. 4D, red, nuclear) where FIG. 4E is an overlay of the images in panels FIG. 4C and FIG. 4D with DAPI nuclear stain (blue) of the same cells. FIG. 4F-FIG. 4H show the same staining strategy for the hippocampus. FIG. 4I shows living skeletal muscle from an Mcm2-EGFP transgenic animal. Accordingly, FIGS. 4A-I demonstrate that EGFP expression from an Mcm2 EGFP transgenic animal occurs where stem cell populations are present. Moreover, colocalizations studies (e.g. FIG. 4C-H) demonstrate that EGFP and Mcm2 are expressed in the same cells.

### EXAMPLE 4

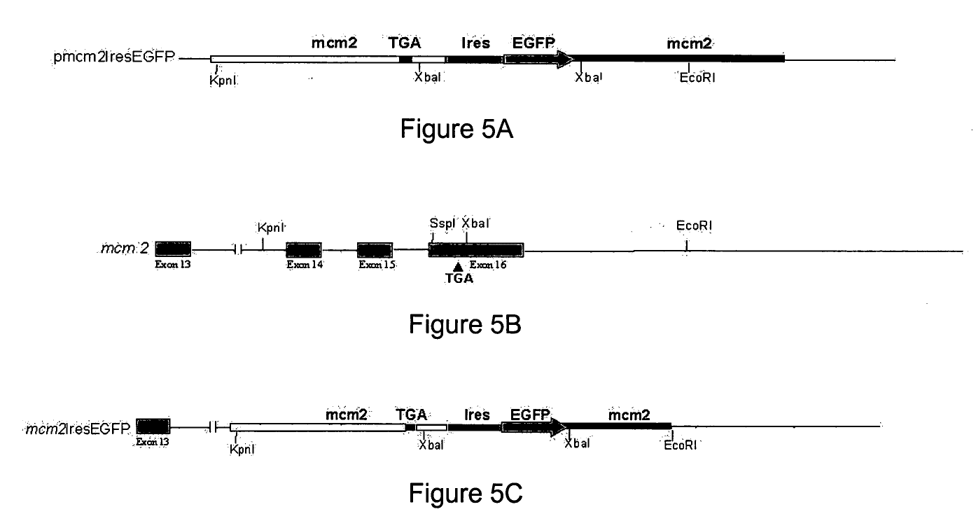

This Example demonstrates the integration of a tamoxifen dependent Cre recombinase transgene (Cre-ERT2) vector useful for driving expression of a reporter transgene in mouse ES cells. The vector is depicted in FIG. 5A and is essentially the same as the vector depicted in FIG. 1A, with the exception of EGFP being replaced by a coding region for tamoxifen dependent Cre recombinase transgene. The vector was electroportated into the mouse ES cell line W4 essentially as described in Example 2. Further modifications include the PgkNeo antibiotic resistance site which enables selection of ES cells that have recombined with the vector. The homologous recombination strategy facilitated by this vector is depicted in FIGS. 5A-5C.

### EXAMPLE 5

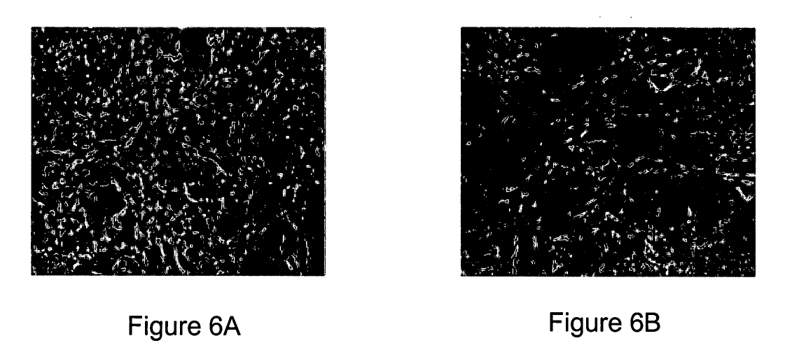

This Example demonstrates a method for using a tamoxifen dependent Cre recombinase (Cre-ERT2) to drive expression of a reporter transgene in mouse ES cells. ES cells and transgenic mice carrying CreERT2 expressed from the Mcm2 gene (Mcm2-IRES-CreERT2) were generated essentially as described in Example 2. The ES cell line identified having the genomic arrangement depicted in FIG. 5C was utilized to confirm that CreERT2 expressed from the Mcm2 promoter can effectively induce lox mediated recombination by transient transfection with a Cre dependent EGFP reporter. Accordingly, Mcm2 -IRES-CreERT2 W4 ES cells were co-transfected with the plasmid constructs Pgk-lox-STOP-lox-EGFP and pcDNA3.1-hcRed as a transfection control and split to media in the presence or absence of tamoxifen. As can be seen from FIG. 6, after the addition of tamoxifen, greater than 90% of hcRed positive cells also expressed EGFP whereas no EGFP expressing cells were found in the absence of tamoxifen. These results demonstrate the generation of ES cells having a homologously recombined CreERT2 that is expressed from the Mcm2 promoter and which allows efficient Cre-mediated reporter transgene activation in the presence of tamoxifen with minimal background in its absence.

### EXAMPLE 6

This Example discloses transgenic mice having an integrated Cre-ERT2 and an integrated tamoxefin inducible Cre-dependent reporter transgene in the form of ROSA-lox-STOP-lox-EGFP. Exposing these mice to tamoxifen will cause activation of Cre recombinase specifically in the stem and proliferative progenitor cells because these cells actively drive transcription from the Mcm2 promoter which in turn transcribes the Cre recombinase transgene coding region. Expression of the Cre recombinase transgene creates permanent genetic rearrangements that are specific to the adult stem cells and their progeny by catalyzing recombination at LOX sites. This Example accordingly discloses mice carrying both the integrated Mcm2-IRES-CreERT2 and the Cre-dependent reporter transgene ROSA-lox-STOP-lox-EGFP. ROSA-lox-STOP-lox-EGFP mice (strain 129-Gt(ROSA)26Sor/J) have been characterized previously for use with Cre dependent reporter transgene expression and were purchased from the Jackson Laboratories (Bar Harbor, ME).

To insert the Mcm2-IRES-CreERT2 transgene in the transgenic mice, essentially the strategy that was successful in creating the Mcm2-IRES-EGFP mice described in Example 3 was used except that the Mcm2-IRES-CreERT2 vector was integrated into the mouse ES cell line W4. A transgenic mouse line in which this targeted recombination was identified has been used to generate both chimeric animals and transgenic animals.

Crossing these transgenic mice with mice having a Cre-dependent reporter transgene such as ROSA-lox-STOP-lox-EGFP will create progeny mice that, after exposure to tamoxefin, will have permanent genetic rearrangements that are specific to the adult stem cells, cancer cells their progeny by catalyzing recombination at LOX sites that allows expression of the reporter transgene.

1. An embryonic stem cell comprising a polynucleotide integrated into a licensing factor gene of the embryonic stem cell wherein the integrated polynucleotide comprises a transgene, wherein the transcription of the transgene is driven by an endogenous promoter of the licensing factor gene, and wherein the integrated transgene is selected from the group consisting of a recombinase and a reporter transgene.

2. The embryonic stem cell of claim 1, wherein the integrated transgene is a reporter transgene.

3. The embryonic stem cell of claim 1, wherein the integrated transgene is a is a fluorescent protein.

4. The embryonic stem cell of claim 3, wherein the reporter transgene encodes a fluorescent protein selected from the group consisting of green fluorescent protein and enhanced green fluorescent protein.

5. The embryonic stem cell of claim 1, wherein the licensing factor gene is selected from the group consisting of ORC genes, Cdc6, Cdt1, Mcm2, Mcm3, Mcm4, Mcm5, Mcm6 and Mcm7.

6. The embryonic stem cell of claim 5, wherein the licensing factor gene is Mcm2.

7. The embryonic stem cell of claim 1, wherein the integrated transgene is a recombinase transgene.

8. The embryonic stem cell of claim 7, wherein the recombinase transgene encodes a recombinase selected from the group consisting of Cre recombinase and tamoxifen inducible Cre recombinase.

9. A cell line derived from the embryonic stem cell of claim 1.

10. A chimeric mouse wherein the genome of at least some of the mouse cells comprises a polynucleotide integrated into a licensing factor gene of the chimeric mouse wherein the integrated polynucleotide comprises a transgene, wherein the transcription of the transgene is driven by an endogenous promoter of the licensing factor gene, and wherein the integrated transgene is selected from the group consisting of a recombinase transgene and a reporter transgene.

11. The chimeric mouse of claim 10, wherein the integrated transgene is a reporter transgene.

12. The chimeric mouse of claim 11, wherein the integrated transgene encodes a fluorescent protein.

13. The chimeric mouse of claim 12, wherein the reporter transgene encodes a fluorescent protein selected from the group consisting of green fluorescent protein and enhanced green fluorescent protein.

14. The chimeric mouse of claim 10, wherein the licensing factor gene is selected from the group consisting of ORC genes, Cdc6, Cdt1, Mcm2, Mcm3, Mcm4, Mcm5, Mcm6 and Mcm7.

15. The chimeric mouse of claim 14, wherein the licensing factor gene is Mcm2.

16. The chimeric mouse of claim 10, wherein the integrated transgene is a recombinase transgene.

17. The chimeric mouse of claim 16, wherein the recombinase transgene encodes a recombinase selected from the group consisting of Cre recombinase and tamoxifen inducible Cre recombinase.

18. A transgenic mouse wherein the genome of each the mouse cells comprises a polynucleotide integrated into a licensing factor gene of the transgenic mouse wherein the integrated polynucleotide comprises a transgene, wherein the transcription of the transgene is driven by an endogenous promoter of the licensing factor gene, and wherein the integrated transgene is selected from the group consisting of a recombinase transgene and a reporter transgene.

19. The chimeric mouse of claim 18, wherein the integrated transgene is a reporter transgene.

20. The chimeric mouse of claim 19, wherein the integrated transgene encodes a fluorescent protein.

21. The chimeric mouse of claim 20, wherein the reporter transgene encodes a fluorescent protein selected from the group consisting of green fluorescent protein and enhanced green fluorescent protein.

22. The chimeric mouse of claim 18, wherein the licensing factor gene is selected from the group consisting of ORC genes, Cdc6, Cdt1, Mcm2, Mcm3, Mcm4, Mcm5, Mcm6 and Mcm7.

23. The chimeric mouse of claim 22, wherein the licensing factor gene is Mcm2.

24. The chimeric mouse of claim 18, wherein the integrated transgene is a recombinase transgene.

25. The chimeric mouse of claim 24, wherein the recombinase transgene encodes a recombinase selected from the group consisting of Cre recombinase and tamoxifen inducible Cre recombinase.

26. A method for making a chimeric mouse wherein the genome of at least some of the mouse cells comprises a polynucleotide integrated into a licensing factor gene of the chimeric mouse wherein the integrated polynucleotide comprises a coding region for a reporter transgene, wherein the transcription of the reporter transgene is driven by an endogenous promoter of the licensing factor gene, the method comprising the steps of:
a) integrating a polynucleotide into a licensing factor gene of a mouse embryonic stem cell wherein the polynucleotide comprises a coding region for a reporter transgene and wherein the transcription of the reporter transgene is driven by a licensing factor gene promoter endogenous to the mouse embryonic stem cell;
b) generating a chimeric mouse embryo comprising the mouse embryonic stem cell from a); and
c) implanting the chimeric mouse embryo into a pseudo-pregnant mouse to obtain a chimeric mouse.

27. The method of claim 26, further comprising the steps of mating the chimeric mouse obtained in c) to another chimeric mouse obtained by the steps a)-c) to produce transgenic offspring.

28. The method of claim 26, wherein the polynucleotide is integrated by homologous recombination.

29. The method of claim 26, wherein the polynucleotide is integrated by gene trapping.

30. The method of claim 26, wherein the integrated transgene is a reporter transgene.

31. The method of claim 30, wherein the integrated transgene encodes a fluorescent protein.

32. The method of claim 31, wherein the reporter transgene encodes a fluorescent protein selected from the group consisting of green fluorescent protein and enhanced green fluorescent protein.

33. The method of claim 26, wherein the licensing factor gene is selected from the group consisting of ORC genes, Cdc6, Cdt1, Mcm2, Mcm3, Mcm4, Mcm5, Mcm6 and Mcm7.

34. The method of claim 33, wherein the licensing factor gene is Mcm2.

35. The method of claim 26, wherein the integrated transgene is a recombinase transgene.

36. The method of claim 35, wherein the recombinase transgene encodes a recombinase selected from the group consisting of Cre recombinase and tamoxifen inducible Cre recombinase.

37. The method of claim 26, further comprising the step of detecting the expression of the reporter transgene to detect stem or cancer cells.

38. The method of claim 27, further comprising the step of detecting the expression of the reporter transgene to detect stem or cancer cells.

39. A method for making a chimeric mouse wherein the genome of at least some of the mouse cells comprises a polynucleotide integrated into a licensing factor gene of the chimeric mouse wherein the integrated polynucleotide comprises a coding region for a recombinase transgene wherein the transcription of the recombinase transgene is driven by an endogenous promoter of the licensing factor gene, the method comprising the steps of:
a) integrating a polynucleotide into a licensing factor gene of a mouse embryonic stem cell wherein the polynucleotide comprises a coding region for a recombinase transgene and wherein the transcription of the recombinase transgene coding region is driven by a licensing factor gene promoter endogenous to the mouse embryonic stem cell;
b) generating a chimeric mouse embryo comprising the mouse embryonic stem cell from a); and
c) implanting the chimeric mouse embryo into a pseudo-pregnant mouse to obtain a chimeric mouse.

37. The method of claim 36, further comprising the steps of mating the chimeric mouse obtained in c) to a transgenic mouse having a recombinase inducible reporter transgene to obtain progeny wherein the expression of the recombinase induces expression of the reporter transgene.

38. The method of claim 36, wherein the polynucleotide is integrated by homologous recombination.

39. The method of claim 36, wherein the polynucleotide is integrated by gene trapping.

40. The method of claim 36, wherein the licensing factor gene is selected from the group consisting of ORC genes, Cdc6, Cdt1, Mcm2, Mcm3, Mcm4, Mcm5, Mcm6 and Mcm7.

41. The method of claim 40, wherein the licensing factor gene is Mcm2.

42. The method of claim 37, wherein the reporter transgene encodes a fluorescent protein.

43. The method of claim 42, wherein the reporter transgene encodes a protein selected from the group consisting of green fluorescent protein and enhanced green fluorescent protein.

44. The method of claim 37, further comprising the step of detecting the expression of the reporter transgene to detect stem or cancer cells.

45. A method of screening for an agent capable of modulating the expression of a licensing factor gene comprising the steps of:
a) administering a test agent to a mouse wherein at least some of the mouse cells comprise a reporter transgene wherein the transcription of the reporter transgene is driven by a licensing factor gene promoter endogenous to the mouse;
b) measuring expression of the reporter transgene;
c) comparing the expression of the reporter transgene in the mouse to which the test agent has been administered with the expression of the reporter transgene in a mouse to which the test agent has not been administered, wherein at least some of the mouse cells of the mouse to which the test agent has not been administered comprise a reporter transgene wherein the transcription of the reporter transgene is driven by a licensing factor gene promoter endogenous to the mouse; and
d) determining whether the expression of the reporter transgene in the mouse to which the test agent has been administered is increased or decreased versus the expression of the reporter transgene in the mouse to which the test agent has not been administered.

46. The method of claim 45, wherein the reporter transgene encodes a fluorescent protein.

47. The method of claim 46, wherein the reporter transgene encodes a protein selected from the group consisting of green fluorescent protein and enhanced green fluorescent protein.

48. The method of claim 45, wherein the licensing factor gene is selected from the group consisting of ORC, Cd6, Cdt1, Mcm2, Mcm3, Mcm4, Mcm5, Mcm6 and Mcm7.

49. The method of claim 48, wherein the licensing factor gene is Mcm2.

50. The method of claim 45, wherein the expression of the reporter transgene is detected by whole body fluorescence imaging.

51. The method of claim 45, wherein all of the nucleated mouse cells comprise a reporter transgene.

52. A method of screening for an agent capable of modulating the expression of a licensing factor gene comprising the steps of:
a) administering a test agent to a mouse wherein at least some of the mouse cells comprise a recombinase transgene wherein the transcription of the recombinase transgene is driven by a licensing factor gene promoter endogenous to the mouse, wherein all of the nucleated mouse cells further comprise a recombinase inducible reporter transgene;
b) inducing the expression of the reporter transgene with the recombinase;
c) measuring expression of the reporter transgene;
d) comparing the expression of the reporter transgene in the mouse to which the test agent has been administered with the expression of the reporter transgene in a mouse to which the test agent has not been administered, wherein at least some of the mouse cells of the mouse to which the test agent has not been administered comprise a reporter transgene wherein the transcription of the reporter transgene is driven by a licensing factor gene promoter endogenous to the mouse; and
e) determining whether the expression of the reporter transgene in the mouse to which the test agent has been administered is increased or decreased versus the expression of the reporter transgene in the mouse to which the test agent has not been administered.

53. The method of claim 52, wherein the reporter transgene encodes a fluorescent protein.

54. The method of claim 53, wherein the reporter transgene encodes a protein selected from the group consisting of green fluorescent protein and enhanced green fluorescent protein.

55. The method of claim 52, wherein the licensing factor gene is selected from the group consisting of ORC, Cdc6, Cdt1, Mcm2, Mcm3, Mcm4, Mcm5, Mcm6 and Mcm7.

56. The method of claim 55, wherein the licensing factor gene is Mcm2.

57. The method of claim 52, wherein the integrated transgene is a recombinase transgene.

58. The embryonic stem cell of claim 57, wherein the recombinase transgene encodes a recombinase selected from the group consisting of Cre recombinase and tamoxifen inducible Cre recombinase.

59. The method of claim 52, wherein the expression of the reporter transgene is detected by whole body fluorescence imaging.
